(19) United States
(12) Patent Application Publication
(10) Pub. No.: US 2014/0270448 A1
(43) Pub. Date: Sep. 18, 2014

(54) SYSTEM AND METHOD FOR ATTENUATION CORRECTION IN EMISSION COMPUTED TOMOGRAPHY

(71) Applicant: UNIVERSITY OF MACAU, Macau (CN)

(72) Inventors: Seng Peng MOK, Macau (CN); Tao SUN, Tianjin (CN)

(73) Assignee: UNIVERSITY OF MACAU, Macau (CN)

(21) Appl. No.: 14/210,457

(22) Filed: Mar. 14, 2014

Related U.S. Application Data
(60) Provisional application No. 61/787,572, filed on Mar. 15, 2013.

(51) Int. Cl. A61B 6/00; A61B 6/03; G06T 11/00

(52) U.S. Cl. CPC A61B 6/5264; G06T 11/005; A61B 6/037
USPC 382/131; 600/538

(57) ABSTRACT

The present invention relates to systems and methods for attenuation correction to improve reconstructed image quality and quantitative accuracy and reduce radiation dose in emission computed tomography. In one embodiment, the present invention provides an interpolated average CT (IACT) method and breathing control devices.

This application claims the benefit of U.S. 61/787,572 filed Mar. 15, 2013. The entire disclosure of the preceding application is hereby incorporated by reference into this application. Throughout this application, various references or publications are cited. Disclosures of these references or publications in their entireties are hereby incorporated by reference into this application in order to more fully describe the state of the art to which this invention pertains.

### FIELD OF THE INVENTION

The present invention relates to systems and methods for attenuation correction to improve reconstructed image quality and quantitative accuracy and reduce radiation dose in emission computed tomography, and relates particularly to systems and methods which employ an interpolated average CT (IACT) method and breathing control devices.

### BACKGROUND OF THE INVENTION

Image quality of emission computed tomography systems such as combined PET/CT or SPECT/CT is hampered by respiratory misalignments and artifacts. In principle, standard helical CT captures a distinct respiratory phase of the thoracic cavity, which does not match with the time-averaged position of the thoracic structures which emission computed tomography, such as PET or SPECT, acquisition captures. The problem of respiratory artifacts has been most closely studied for myocardial perfusion PET/CT, while more than 40% of the studies have artifactual defects in the cardiac region when no steps are taken to address the PET/CT alignment, causing significant diagnostic error (Gould et al., 2007). Different types of mismatch led to artifacts and increases in myocardial nonuniformity (McQuaid et al., 2008; Loghin et al., 2004). Misregistration of the stress PET/CT also affected the global and regional myocardial blood flow estimation (Martinez-Möller et al., 2007; Rajaram et al., 2013). In some studies, underestimation of the standardized uptake value (SUV) of the lung lesions was observed (Erdi et al., 2004; Nehmeh et al., 2008; Adams et al., 2010). Mismatched attenuation correction (AC) can also cause SUV overestimation for lower lung tumors that are located close to the liver dome, leading to complicated SUV errors (Liu et al., 2009). All these PET/CT mismatching artifacts will lead to inaccurate localization and quantification of tumors, hence potential misdiagnoses (Osman et al., 2003; Allen-Auerbach et al., 2006; von Schulthess et al., 2006; Pan et al., 2013).

Besides simple manual or automatic registration based on the outline of the heart for the misaligned PET and CT as suggested by Martinez-Möller et al. (2007), several methods have been investigated mostly to reduce PET/CT misalignments and artifacts: breathing instruction, CT protocols, and gated four-dimensional (4D) PET/CT. Breathing instruction methods, like normal end-expiration breath-hold during CT scan, reduce the occurrence and the severity of respiratory curvilinear artifacts on co-registered PET/CT images (Goerres et al., 2002). Juan et al. (2004) showed that PET reconstructed images from patients of normal end-expiration breath-hold group had 28% less incidence rate of artifacts as compared with those from the free-breathing group. This method is not practical for all patients since it requires patients' compliance and may not be feasible for patients with limited pulmonary function (Senan et al., 2004). Specific CT protocols based on the axial or helical mode have also been proposed for AC in PET images. Low-pitch CT approximates the average respiratory position and may introduce some blurriness which matches the conditions that occur during PET measurement (Nye et al., 2007). Cine average CT (CACT) was developed for AC in PET and showed significantly less misalignments and artifacts as compared with conventional helical CT (HCT)-based AC (Pan et al., 2005; Pan et al., 2006). Alessio et al. (2007) further evaluated both average and intensity maximum images of 4D CT and indicated the later method had better alignments between PET and CT. The main problem of CACT is relatively high radiation dose.

Gated 4D PET/CT provides possible motion compensation in PET reconstruction and motion information for radiation therapy (Nehmeh et al., 2004). In 4D PET/CT, each respiratory PET bin can be transformed to a reference target frame that corresponds well to a matched HCT or a CT frame acquired from 4D CT data. All registered bins were used to form a single PET bin for AC to improve quantitative accuracy (Nagel et al., 2006; Wells et al., 2010; McQuaid et al., 2011; Didierlaurent et al., 2012). Recently Fayad et al. (2013) proposed to generate a virtual 4D CT based on one reference CT image and 4D motion fields obtained from PET to reduce the radiation dose. Liu et al. (2010) developed another quiescent period gating method to utilize the end-expiration quiescent phase of PET data to match the end-expiration CT. Some investigators incorporated motion estimation into the iterative reconstruction process to lower image noise of gated PET bins which had less photon counts (Lamare et al., 2007; Grotus et al., 2009). However, its main disadvantages are the high dose from the potential 4D CT, increased acquisition and postprocessing time. More details on different respiratory artifact reduction techniques are described in Sun et al., 2012.

Hence, there is a need for reducing respiratory misalignments and artifacts and improving quantitative accuracy in emission computed tomography images such as PET or SPECT by using specialized low dose CT images as attenuation map.

### SUMMARY OF THE INVENTION

The present invention provides a method for attenuation correction in reconstructed emission computed tomography (ECT) images. In one embodiment, said method comprises: (i) generating Interpolated Average CT (IACT) image of a subject based on one or more CT images acquired during momentary suspension of breathing motion at specific phases in the breathing cycle of a subject by means of a breathing control device; (ii) acquiring an ECT image from the same section of the subject; and (iii) correcting attenuation in said ECT image of step (ii) by using said IACT image generated previously from step (i) as an attenuation map in the ECT reconstruction that comprises the use of a reconstruction algorithm, thereby resulting in reconstructed ECT images with attenuation correction.

The present invention also provides a method for generating an Interpolated Average CT image. In one embodiment, said Interpolated Average CT image is generated by: (a) suspending breathing motion of said subject momentarily at said one or more specific phases in a breathing cycle by means of said breathing control device; (b) acquiring one or more CT images of said subject when the breathing motion is suspended by said breathing control device; (c) obtaining a deformation matrix of said CT images of step (b) by deformable image registration; (d) interpolating between the images of step (b) to obtain intermediate images base on said deformation matrix of step (c); and (e) generating Interpolated Average CT images by averaging the intensity of the images of step (b) and the intermediate images of step (d).

The present invention further provides a system for generating Interpolated Average CT image of a subject. In one embodiment, said system comprises: (i) an active breathing controller (ABC) for momentarily suspending breathing motion of a subject at one or more specific phases in a breathing cycle, said ABC comprises a flow sensor, a valve, a microcontroller and an airtube system, wherein said subject breathes through the airtube system; (ii) a computing device comprising a program for identifying one or more specific phases in the breathing cycle based on breathing flow rate data, said device is configured to control the valve in the ABC; (iii) a CT scanner, wherein said CT scanner acquires one or more CT images when said subject's breathing motion is momentarily suspended by said ABC.

### DEFINITIONS & ABBREVIATIONS

The following terms shall be used to describe the present invention. In the absence of a specific definition set forth herein, the terms used to describe the present invention shall be given their common meaning as understood by those of ordinary skill in the art.

As used herein, the expression “ABC” refers to Active Breathing Controller.

As used herein, the expression “AC” refers to Attenuation Correction.

As used herein, the expression “CT” refers to Computed Tomography.

As used herein, the expression “CACT” refers to Cine Average Computed Tomography.

As used herein, the expression “IACT” refers to Interpolated Average Computed Tomography.

As used herein, the expression “IACT-ABC” refers to IACT obtained with the use of an ABC.

As used herein, the expression “HCT” refers to Helical Computed Tomography.

As used herein, the expression “ECT” refers to Emission Computed Tomography.

As used herein, the expression “PET” refers to Positron Emission Tomography.

As used herein, the expression “SPECT” refers to Single Photon Emission Computed Tomography.

As used herein, the expression “PET/CT” refers to Positron Emission Tomography coupled with Computed Tomography.

As used herein, the expression “SPECT/CT” refers to Single Photon Emission Computed Tomography coupled with Computed Tomography.

As used herein, the expression “PET_{HCT}” refers to PET images reconstructed using HCT as attenuation map.

As used herein, the expression “PET_{HCT-in}” refers to PET images reconstructed using HCT acquired at the end-inspiration phase of a breathing cycle as attenuation map.

As used herein, the expression “PET_{HCT-ex}” refers to PET images reconstructed using HCT acquired at the end-expiration phase of a breathing cycle as attenuation map.

As used herein, the expression “PET_{IACT}” refers to PET images reconstructed using IACT as attenuation map.

As used herein, the expression “HCTᵢₙ” refers to Helical Computed Tomography acquired at the end-inspiration phase of a breathing cycle.

As used herein, the expression “HCTₑₓ” refers to Helical Computed Tomography acquired at the end-expiration phase of a breathing cycle.

As used herein, the expression “Φᵢₑ” refers to forward deformation vector calculated from end-inspiration phase to end-expiration phase.

As used herein, the expression “Φₑᵢ” refers to backward deformation vector calculated from end-expiration phase to end-inspiration phase.

As used herein, the expression “d_{HCT/PET}_{_{HCT}}” refers to quantitative measurement of the centroid difference between HCT and PET_{HCT}.

As used herein, the expression “d_{IACT/PET}_{_{IACT}}” refers to quantitative measurement of the centroid difference between IACT and PET_{IACT}.

As used herein, the expression “SUV” refers to Standardized Uptake Value.

As used herein, the expression “SUVₘₐₓ” refers to maximum SUV value.

As used herein, the expression “SUVₘₑₐₙ” refers to mean SUV value.

As used herein, the expression “TBR” refers to target-to-background ratio.

As used herein, the expression “LLL” refers to lower left lung.

As used herein, the expression “LRL” refers to lower right lung.

As used herein, the expression “MRL” refers to middle right lung.

As used herein, the expression “URL” refers to upper right lung.

As used herein, the expression “IR” refers to intensity ratio.

As used herein, the expression “VOI” refers to volume-of-interest.

As used herein, the expression “ROI” refers to Region-of-interest.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (a) shows the overview and (b) block diagram of one embodiment of the ABC system to generate IACT for AC in emission computed tomography.

FIG. 2 shows the change of respiratory signals during externally mediated (a) end-inspiration and (b) end-expiration breath-hold controlled by an embodiment of the ABC of this invention; arrows indicated the period for which the valve is closed and patients' breathing is suspended.

FIG. 3 shows the deformation fields (Φᵢₑ and Φₑᵢ) obtained from B-spline method that were used to generate the interpolated images for IACT based on an empirical sinusoidal function in one embodiment of the present invention.

FIG. 4 shows the transaxial (left), coronal (middle), and sagittal (right) views of the fused PET/CT images for (a) HCT- and (b) IACT-AC for patient #11; arrows indicate a lesion in the right upper lobe of the lung.

FIG. 5 shows sample coronal images of (a) HCT (left); PET_{HCT} (middle); fusion image (right) and (b) IACT (left); PET_{IACT} (middle); fusion image (right) for patient #10. Artifacts around the left diaphragm and left ventricle region were observed on the PET_{HCT} images (black arrows). (c) Vertical image profiles drawn across the lesion on PET_{HCT} and PET_{IACT} images; solid arrow indicates the lesion area and the dotted arrow indicates the liver region.

FIG. 6 shows the coronal views of (a) PET_{HCT} and (b) PET_{IACT} for patient #13; curvilinear artifact was observed on PET_{HCT} (arrow) but reduced on PET_{IACT}.

FIG. 7 shows sample respiratory signal of a patient. The arrow indicated residual breathing during a breath hold mediated by one embodiment of the ABC of the present invention.

FIG. 8 shows (a) normal end-inspiration breath-hold CT and (b) normal end-expiration breath-hold CT captured with the use of ABC.

FIG. 9 shows sample images for subject #1; (a) conventional helical CT (HCT); (b) IACT; (c) the associated PET AC images of FIG. 9 (a); (d) the associated PET AC images of FIG. 9 (b).

FIG. 10 shows image profiles for HCT- and IACT-based AC images in FIG. 9 (b); the arrow indicates substantial difference between two PET images using different AC methods.

FIG. 11 shows the simulated attenuation maps representing (a) CACT; (b) IACT; (c) HCTᵢₙ; and (d) HCTₑₓ.

FIG. 12 shows the simulated noise-free average activity maps showing a 20 mm thoracic lesion with TBR of 4:1 placed at (a) LLL, (b) LRL, (c) MRL and (d) URL.

FIG. 13 shows the sampled PET reconstructed images with AC using CACT. Upper and lower circles indicated the ROIs drawn on the LRL lesion and background respectively.

FIG. 14 shows the sampled coronal PET reconstructed images with LRL lesion using different AC maps; arrows indicate the misalignment artifacts when using HCTs for AC.

FIG. 15 shows the TBR for 20 mm lesions located at (a) LLL (b) LRL (c) MRL and (d) URL for a standard TBR of 4:1 and respiratory amplitude of 2 cm.

FIG. 16 shows the TBR for 20 mm lesions located at (a) LLL (b) LRL (c) MRL and (d) URL for a standard TBR of 8:1 and respiratory amplitude of 2 cm.

FIG. 17 shows the TBR for 10 mm lesions located at (a) LLL (b) LRL (c) MRL and (d) URL for a standard TBR of 8:1 and respiratory amplitude of 2 cm.

FIG. 18 shows the TBR for 20 mm lesions located at (a) LLL (b) LRL (c) MRL and (d) URL for a standard TBR of 6:1 and respiratory amplitude of 3 cm.

FIG. 19 shows the TBR for 20 mm lesions located at (a) LLL (b) LRL (c) MRL and (d) URL for a standard TBR of 12:1 and respiratory amplitude of 3 cm.

FIG. 20 shows the TBR for 10 mm lesions located at (a) LLL (b) LRL (c) MRL and (d) URL for a standard TBR of 12:1 and respiratory amplitude of 3 cm.

FIG. 21 shows the various attenuation maps used in Example 4; (a) Average ¹⁸F-FDG activity map; (b) Respiratory phase#1 of the attenuation map represented helical CT obtained at end-inspiration (HCT-1); (c) Respiratory phase#8 of the attenuation map represented helical CT obtained at end-expiration (HCT-8); (d) Average attenuation map represented CACT; (e) IACT.

FIG. 22 shows the short-axis images showing lesion placed at (a) lateral wall and (b) inferior wall.

FIG. 23 shows the bull's eye plots generated from PET short-axis reconstructed images with normal cardiac uptake using different AC schemes for motion amplitude of (a) 2 cm, (b) 3 cm and (c) 4 cm.

FIG. 24 shows the bull's eye plots generated from PET short-axis reconstructed images with a lesion located at the inferior wall using different AC schemes for motion amplitude of (a) 2 cm, (b) 3 cm and (c) 4 cm.

FIG. 25 shows the bull's eye plots generated from PET short-axis reconstructed images with a lesion located at the lateral wall using different AC schemes for motion amplitude of (a) 2 cm, (b) 3 cm and (c) 4 cm.

FIG. 26 shows the circumferential profiles at a distance of apex to base of the bull's eye plots using different AC schemes for motion amplitude of (a) 2 cm, (b) 3 cm and (c) 4 cm. Circumferential profiles at a distance of 2/4 from the total distance of apex to base of the bull's eye plots using different AC schemes for motion amplitude of (d) 2 cm, (e) 3 cm and (f) 4 cm.

FIG. 27 shows the bull's eye plots from different AC maps from three patients.

FIG. 28 shows the circumferential profiles from three patients.

FIG. 29 shows a chart comparing the effective dosage required from different CT protocols.

FIG. 30 shows the simulation results for ⁸²Rb perfusion study; the simulated bull's eye plot using IACT for AC was more similar to the one using CACT for AC, as also indicated in the profiles generated from different apex-to-base distances.

### DETAILED DESCRIPTION OF THE INVENTION

The present invention provides a method for attenuation correction in reconstructed emission computed tomography (ECT) images. In one embodiment, said method comprises: (i) generating Interpolated Average CT (IACT) image of a subject based on one or more CT images acquired during momentary suspension of breathing motion at specific phases in the breathing cycle of a subject by means of a breathing control device; (ii) acquiring an ECT image from the same section of the subject; and (iii) correcting attenuation in said ECT image of step (ii) by using said IACT image generated previously from step (i) as an attenuation map in the ECT reconstruction that comprises the use of a reconstruction algorithm, thereby resulting in reconstructed ECT images with attenuation correction.

In one embodiment, said emission computed tomography is Positron Emission Tomography (PET) or Single Photon Emission Computed Tomography (SPECT).

In one embodiment, said specific phase in the breathing cycle comprises one or more of end-expiration phase, end-inspiration phase and any one of the mid-respiratory phases.

In one embodiment, said image reconstruction algorithm is a 2-D, 2.5-D, 3-D or 4-D image reconstruction algorithm is selected from the group consisting of Ordered Subsets Expectation Maximization (OS-EM) image reconstruction algorithm, Filtered Back Projection (FBP) method, Maximum Likelihood Expectation Maximization (ML-EM), Maximum a Posteriori Expectation Maximization (MAP-EM) with different priors, Maximum a Posteriori Expectation Maximization based on a One-Step-Late algorithm (MAP-EM (OSL)) with different priors, Row Action maximum Likelihood Algorithm (RAMLA), 3D ReProjection (3DRP), Single Slice Rebinning (SSRB), Fourier Rebinning (FORE), Fourier Rebinning with 2D algorithm (FORE+2D), and Fourier Rebinning with Average-Weighted OS-EM Expectation Maximization (FORE+AWOS-EM).

In one embodiment, said subject has one or more lesions or no lesions in the thoracic cavity.

In one embodiment, said reconstructed image is used in assessing cardiac viability, myocardial perfusion, or presence or quantification of lesions in said subject.

The present invention also provides a method for generating an Interpolated Average CT image. In one embodiment, said Interpolated Average CT image is generated by: (a) suspending breathing motion of said subject momentarily at said one or more specific phases in a breathing cycle by means of said breathing control device; (b) acquiring one or more CT images of said subject when the breathing motion is suspended by said breathing control device; (c) obtaining a deformation matrix of said CT images of step (b) by deformable image registration; (d) interpolating between the images of step (b) to obtain intermediate images base on said deformation matrix of step (c); and (e) generating Interpolated Average CT images by averaging the intensity of the images of step (b) and the intermediate images of step (d).

In one embodiment, said interpolation of step (d) is linear or nonlinear interpolation.

In one embodiment, said nonlinear interpolation is based on the movement function of an internal organ during respiration, defined by: z(t)=zₒ−b cos²ⁿ(πt/τ) where z(t)=position of said organ at time, t; zₒ=organ position at end-expiration; b=amplitude of motion; τ=period of motion; n=degree of symmetry which depends on the patient-specific respiratory signal. In another embodiment, said internal organ is liver or diaphragm of said subject.

In one embodiment, said nonlinear interpolation is based on respiratory signals generated by a computer simulation model. In another embodiment, said computer simulation model is a 4-Dimensional Non-uniform Rational B-spline (NURBS) based Cardiac-Torso (XCAT) phantom.

In one embodiment, said nonlinear interpolation is based on read-in patient-specific respiratory signal.

In one embodiment, said breathing control device of step (a) is an active breathing controller.

In one embodiment, said CT image of step (b) is acquired using helical CT or cine CT.

In one embodiment, said CT image of step (b) is acquired at a reduced radiation dose. In another embodiment, said radiation dosage is reduced up to 85% as compared to conventional helical CT.

The present invention further provides a system for generating Interpolated Average CT image of a subject. In one embodiment, said system comprises: (i) an active breathing controller (ABC) for momentarily suspending breathing motion of a subject at one or more specific phases in a breathing cycle, said ABC comprises a flow sensor, a valve, a microcontroller and an airtube system, wherein said subject breathes through the airtube system; (ii) a computing device comprising a program for identifying one or more specific phases in the breathing cycle based on breathing flow rate data, said device is configured to control the valve in the ABC; (iii) a CT scanner, wherein said CT scanner acquires one or more CT images when said subject's breathing motion is momentarily suspended by said ABC.

In one embodiment, said airtube system comprises a mask or mouth piece for said subject to breathe through.

In one embodiment, said flow sensor measures breathing flow rate of said subject breathing through the airtube system.

In one embodiment, said microcontroller receives breathing flow rate signal from the flow sensor and sends the signal to the computing device.

In one embodiment, said computing device closes the valve in the ABC to suspend breathing motion of said subject when one or more specific phases in the breathing cycle is identified manually or automatically by said program.

In one embodiment, CT images for generating Interpolated Average CT image are acquired manually or automatically when the breathing motion of said subject is suspended.

In one embodiment, said CT scanner is coupled with a PET or a SPECT scanner.

The invention will be better understood by reference to the EXAMPLES which follow, but those skilled in the art will readily appreciate that the specific experiments detailed are only illustrative, and are not meant to limit the invention as described herein, which is defined by the claims which follow thereafter.

### Example 1

### Active Breathing Controller (ABC) Design

The ABC used in this example integrates a spirometer, an air mask/mouth piece, and a tube-valve system as shown in FIG. 1. Patients were asked to perform mouth-breathing using the air mask or mouth piece that is connected to the tube. The flow sensor inserted in the tube detected real-time breathing flow rate of the patients and sampled the signal to the microcontroller, which further preprocessed the signal and sent it to a computer through a USB connector. An acquisition program based on C++ was developed to process the input signal and control the switching of the valve in the tube. The program can detect the change of the air flow direction according to the flow rate measurement to locate the end-inspiration and end-expiration phases, while the trigger circuit can then automatically control the closing of the valve located in the end of tube to suspend the patients' breathing. Hence, the operator only needs to notify the program when he/she is ready to acquire the CT data. The flow rate signal can be integrated to determine the change of lung volume during the respiratory cycle. Before the acquisition a system calibration was performed in order to compensate signal drift caused by substantial change of temperature and atmospheric pressure. Subjects were coached before the actual CT scans to adapt to the ABC. The operator manually started the HCT scans once the patient had demonstrated the ability to hold his/her breathe for ˜6 s during the two extreme phases. Sample changes of lung volume during the breathing cycles under the ABC control are shown in FIG. 2.

### Patient Population

Between October 2012 and December 2012, 15 patients were recruited with a total of 18 lesions in different thoracic regions: left upper lobe (2), right upper lobe (4), right hilum (3), right lower lobe (3), left hilum (2), and esophagus (4). A summary of the demographic data of the patients is shown in Table I. This study was approved by the Institutional Review Board of Taipei Veterans General Hospital, and written informed consent was obtained from all patients. All patients were recruited through their scheduled whole-body PET/CT procedures, and those who had history of inferior pulmonary function or were unable to follow the breath-hold procedures were excluded from this study (n=3).

TABLE I
Patient demographic data.

| Patient | | Age | | Lesion volume |
|---|---|---|---|---|
| no. | Sex | (yr) | Lesion location | (c.c.) |
| 1 | F | 65 | Left upper lobe | 5.29 |
| 2 | F | 66 | Right upper lobe | 8.28 |
| 3 | M | 86 | Right middle lobe, close to rib | 3.45 |
| 4 | M | 81 | Right middle lobe | 21.64 |
| 5 | M | 59 | Left upper lobe | 7.63 |
| 6 | M | 51 | Right upper lobe | 2.15 |
| | | | Right lower lobe | 4.54 |
| | | | Left upper lobe, close to rib | 1.3 |
| 7 | M | 59 | Right middle lobe | 1.45 |
| | | | Left hilum | 5.56 |
| 8 | M | 66 | Right hilum | 1.6 |
| 9 | M | 72 | Right upper lobe | 7.09 |
| 10 | M | 88 | Right upper lobe, close to rib | 9.53 |
| 11 | M | 68 | Right middle lobe | 11.39 |
| 12 | M | 61 | Esophagus | 29.6 |
| 13 | M | 47 | Esophagus | 25.38 |
| 14 | M | 42 | Esophagus | 4.79 |
| 15 | M | 60 | Esophagus | 15.48 |

### Acquisition Protocol

Patients were injected with 300-480 MBq of ¹⁸F-FDG, the relative dose measured according to each patient's weight and scanned 1 h post injection. For each patient, four imaging sessions were acquired:
(i) standard shallow free breathing whole-body HCT [120 kV, smart mA (range 30-150 mA), helical mode, 0.984:1 pitch, 0.5 s gantry rotation];
(ii) whole-body PET for seven bed positions with 3 min/bed;
(iii) end-inspiration and end-expiration breath-hold HCTs aided with ABC (120 kV, 10 mA helical mode, 0.984:1 pitch, 0.5 s gantry rotation time and a total of 4.4 s/scan) for the thoracic region;
(iv) thoracic PET for two bed positions with 3 min/bed.

All scans were acquired using a PET/CT scanner (Discovery VCT, GE Medical Systems, Milwaukee, Wis.) in three dimensional (3D) mode with transaxial field-of-views (FOVs) of 70 and 50 cm for PET and CT, respectively. The thoracic PET was performed due to the fact that the patients changed position after the whole body session for accommodating the ABC device.

### IACT Generation

B-spline, a deformable image registration algorithm, was applied to calculate the deformation vectors which includes lateral, anterior-posterior, and inferior-superior displacement for each voxel on two CT volumes, i.e., end-inspiration and end-expiration phases obtained from ABC (Sarrut et al., 2006), based on the Insight Segmentation and Registration Toolkit (ITK) (Yoo et al., 2002). One CT image was chosen as the fixed image, i.e., end-inspiration phase, while the other was used as the moving image, i.e., end-expiration phase. A single rigid registration was conducted in the first step. Three stages of B-spline registration were performed later using a multi-resolution method in the second step. The grid resolution of the control points improved and their grid-spacing decreased along different stages in this step. The deformation field was determined when the mean square error of the two CT images was smaller than a positive value ε (0.001) in each resolution level. The forward deformation vector Φᵢₑ was calculated from end-inspiration phase #1 to end-expiration phase #7 and the backward deformation vector Φₑᵢ was calculated from phase #7 to phase #1. For interpolation of Φᵢₑ and Φₑᵢ to obtain the interpolated phases, a diaphragmatic movement function during respiratory cycles was introduced (Lujan et al., 2003):

$$z ( t ) = {z}_{o} - b {cos}^{2 n} ( \frac{π t}{τ} ) , \qquad Eqn . ( 1 )$$

where z(t)=position of diaphragm at time t, zₒ=diaphragm position at end-expiration, b=amplitude of motion, τ=period of motion, and n=degree of asymmetry which depends on the patient-specific respiratory signals. The power of “2n” made Eqn. (1) resulted in more phases near the end-expiration than near the end-inspiration. The inspiration/expiration ratios were calculated for one respiratory cycle for different integer n from Eqn. (1) and compared with the actual ratios measured in the acquired breathing signal from each patient, and n was determined when its corresponding ratios had the best fit with the measured data. In this example, n=3 was used for most patients except n=2 for patient #7 and n=4 for patient #12. To generate intermediate images, Φᵢₑ was divided based on z(t) to obtain interpolated deformation fields. Thus, interpolated phases #2, #3, #4, #5, and #6 were generated by warping original phase #1 based on Φᵢₑ (FIG. 3). Similarly, phases #8, #9, #10, #11, and #12 were warped from phase #7 based on Φₑᵢ. The final IACT was generated by averaging the image intensity of ten interpolated and two original extreme phases, i.e., a total of 12 phases.

### Data Postprocessing

The PET raw data were reconstructed using 3D ordered subset expectation maximization (OS-EM) (28 subsets; 2 iterations; pixel matrix of 128×128) algorithm available on the PET/CT workstation. The PET sinograms were corrected for random, scatter, isotope decay and attenuation using HCT and IACT, respectively. The PET reconstructed images with AC using HCT and IACT, i.e., PET_{HCT} and PET_{IACT}, were registered with associated CT for further analysis.

### Image Analysis

### Misalignments Between PET and CT

A 3D volume-of-interest (VOI) was delineated for each lesion on PET images using a semiautomatic region growing method with a 50% cut off threshold of the maximum intensity value (Boellaard et al., 2010; Mah et al., 2002; Deniaud-Alexandre et al., 2005). The corresponding delineation for VOI in the CT images was performed with the “lung” window by a radiation oncologist. The coordinates of the centroid of the lesion in PET_{HCT}, HCT, PET_{IACT}, and IACT were determined on the chosen VOIs (Nehmeh et al., 2007; Fin et al., 2008). The distances (d) between the lesion centroid of PET and associate CT were then obtained.

### Standardized Uptake Value (SUV)

For each lesion, maximum SUV value (SUVₘₐₓ) and mean SUV value (SUVₘₑₐₙ) were measured based on the VOIs drawn on the PET_{HCT} and PET_{IACT} images.

### Radiation Dose

For different CT protocols and subjects, the radiation dose was expressed using the effective dose in mSv for the thoracic region. An approximation of the effective dose was obtained by multiplying the volume CT dose length product (mGy-cm) as reported by the manufacturer's software with a conversion factor k (0.014 mSv mGy-1 cm-1) (Bongartz et al., 2004).

### Results

For all 15 patients with 18 lesions, IACT generally reduced lesion mismatch (d) between CT and corresponding PET attenuation corrected images (Table II), with average decrease of 1.34±1.79 mm among all measurable lesions. The centroid difference was not obtainable for patients with esophageal lesions as they could not be delineated on the CT images.

Meanwhile, the SUVₘₐₓ and SUVₘₑₐₙ for the lesions are summarized in Table II. PET_{IACT} generally showed increased SUVₘₐₓ and SUVₘₑₐₙ for all lesions when compared to PET_{HCT}. The percentage increments (% diff) are (30.95±18.63)% and (22.39±15.91)% for SUVₘₐₓ and SUVₘₑₐₙ, respectively.

Sample images of three patients with lung lesions are shown in FIGS. 4 to 6, respectively. In FIG. 4, the IACT AC method provided a better matching for the CT and PET image as compared to HCT for AC. The visual assessment matched with the quantitative measurement of the centroid difference between CT and PET in Table II (d_{HCT/PET}_{_{HCT}}=9.34 mm; d_{IACT/PET}_{_{IACT}}=1.36 mm; diff=−7.98 mm). For another patient as demonstrated in FIG. 5, the contrast of the lesion in the right upper lobe substantially improved for PET_{IACT} as compared to PET_{HCT}, with increase of SUVₘₐₓ and SUVₘₑₐₙ of 53.8% and 30.61%, respectively. The general resolution also improved for PET_{IACT} as compared to PET_{HCT} in this patient as indicated in the vertical profiles drawn on FIG. 5(c), where the lesion full-width-at-half-maximum appeared to be smaller with improved structure details in the liver region on PET_{IACT}. The PET_{HCT} had severe “cold” artifacts around the left ventricle region and also in the region close to the left diaphragm, while the artifacts were reduced in the PET_{IACT}. Characteristic curvilinear artifact was also observed on PET_{HCT} in patient #13 (FIG. 6). The estimated effective dose was 0.38 mSv for IACT for the thoracic region, while HCT had an average effective dose of 2.1 mSv (1.58-2.42 mSv) in the same region. The IACT reduced up to 84.29% effective dose as compared to HCT method in this example.

TABLE II
Summary of different quantitative figures-of-merit for HCT and IACT AC methods

| | SUVₘₐₓ | | | SUVₘₑₐₙ | | | d (mm) | | |
|---|---|---|---|---|---|---|---|---|---|
| Patient# | PET_{HCT} | PET_{IACT} | % diff | PET_{HCT} | PET_{IACT} | % diff | HCT/PET_{HCT} | IACT/PET_{IACT} | Diff. (mm) |
| 1 | 5.98 | 7.01 | 17.22 | 2.48 | 2.78 | 12.1 | 6.28 | 5.91 | −0.37 |
| 2 | 7.99 | 9.07 | 13.52 | 3.89 | 5.81 | 49.36 | 3.79 | 6.49 | 2.7 |
| 3 | 4.64 | 6.26 | 33.84 | 2.89 | 2.98 | 3.11 | 6.6 | 4.67 | −1.93 |
| 4 | 12.03 | 14.97 | 24.44 | 4.96 | 5.75 | 15.93 | 4.68 | 4.59 | −0.09 |
| 5 | 3.27 | 5.67 | 73.39 | 1.23 | 1.8 | 46.34 | 7.32 | 6.22 | −1.1 |
| 6 | 6.39 | 7.91 | 23.79 | 3.77 | 4.83 | 28.12 | 4.74 | 1.97 | −2.77 |
| | 2.47 | 2.67 | 8.1 | 1.32 | 1.52 | 15.15 | 6.58 | 4.47 | −2.11 |
| | 3.5 | 4.35 | 24.29 | 2.22 | 2.77 | 24.77 | 13.77 | 8.79 | −4.98 |
| 7 | 1.98 | 2.77 | 39.9 | 1.2 | 1.91 | 59.17 | 5.27 | 4.39 | −0.88 |
| | 2.69 | 3.29 | 20.07 | 1.51 | 1.53 | 1.32 | 12.12 | 14..67 | 2.55 |
| 8 | 1.53 | 2.35 | 53.59 | 1.01 | 1.11 | 9.9 | 6.55 | 6.47 | −0.88 |
| 9 | 7.84 | 8.46 | 7.91 | 3.96 | 4.57 | 15.4 | 2.93 | 0.57 | −2.36 |
| 10 | 1.84 | 2.83 | 53.8 | 1.47 | 1.92 | 30.61 | 4.91 | 5.53 | 0.62 |
| 11 | 7.65 | 10.71 | 40 | 5.32 | 6.75 | 26.88 | 9.34 | 1.36 | −7.98 |
| 12 | 15.77 | 21.3 | 35.07 | 9.3 | 11.75 | 26.34 | | | |
| 13 | 4.77 | 4.98 | 4.4 | 3.23 | 3.66 | 15.48 | | | |
| 14 | 8.12 | 10.85 | 33.62 | 5.21 | 5.77 | 10.75 | N/A (Lesions cannot not be on CT | | |
| | | | | | | | images) | | |
| 15 | 6.27 | 9.41 | 50.08 | 3.69 | 4.14 | 12.2 | | | |
| Mean | 5.82 | 7.49 | 30.95 | 3.26 | 3.96 | 22.39 | 6.78 | 5.44 | −1.34 |
| SD | 1.94 | 2.21 | 18.63 | 1.45 | 1.62 | 15.91 | 3.07 | 3.46 | 2.79 |

### Discussion

The ABC device in this example samples the respiratory signal by detecting the air flow with 20 Hz sampling frequency, while the peaks and the valleys of the signal indicates end-inspiration and end-expiration phases, respectively. During the acquisition, the operator observes the real time signal and indicates when he/she is ready for acquiring the CT data. The acquisition program will then automatically determine the extreme phases and send a high level voltage signal to the electronic board to close the valve connected to the breathing tube with <0.1 s. The patient will hear a sharp sound from the closing valve and a backward force from the tube will then prompt the patient to hold his/her breath. The time from the valve closing to patient's response lasts <1 s. The closing time of the valve was set to ˜6 s to enable patients to hold their breath sufficiently long enough for the HCT scan, which takes ˜4.4 s. The short breath-holding time particularly enhances the feasibility of this invention, as compared with other breathing instruction methods (Nehmeh et al., 2007). Simulation study showed that the IACT generated from the “shifted” respiratory phases, i.e., the phases right after the extreme phases, provided results very similar to the IACT generated from the exact end-inspiration and end-expiration phases. Besides, some patients may have residual breathing after the valve is closed (FIG. 7), which may affect the subsequent IACT generation. Thus, besides coaching, all patients underwent a mock acquisition before the experiment to confirm their compliance of breath-holding.

A tube current of 10 mA was used for the IACT acquisition because it was the lowest value available on the current scanner. Even though the 10 mA CT images are much nosier than the conventional diagnostic CT studies, they are still feasible for generating the IACT for AC in PET/CT. While IACT only imposed 0.38 mSv dose from two separate low dose HCT scans to each patient, the improvement of image quality and quantitative accuracy was substantial. According to Xia et al., 2012, the PET noise introduced by the ultralow dose CT scans does not significantly affect the diagnostic information and the image quality of the associate attenuation-corrected PET. Hence, CT dose can be reduced while it is mainly for AC and providing registered anatomical information, but may not be feasible for diagnostic purpose. Notably, the IACT for PET AC is relatively smoothed by averaging the extreme and interpolated images. Thus, extra smoothing for CT raw data was not performed before the IACT generation.

The movement of the structures in the thorax is highly correlated to the diaphragm motion during respiration (Gervino et al., 2009). Thus, a diaphragmatic movement function [Eq. (1)] during the respiratory cycles was used to model the lesion movement pattern. On the other hand, the B-spline method used in this example estimated the deformation vector of the lesion in three directions: superior-to-inferior, anterior-to posterior, and lateral. However, this combined vector may not indicate hysteresis motion when the lesion moving trajectories are not consistent from inspiration to expiration and vice versa. This motion will induce a larger tumor volume size appeared in the PET images as compared to its real size, leading to PET/CT misalignment. The lesions in the upper lung are more subject to respiratory hysteresis and are more complicated to model as compared to the ones in the lower lung (Seppenwoolde et al., 2002; Boldea et al., 2008). In this example, only some of the lesions in the upper lung were observed to have inferior mismatching in IACT-AC as compared to using HCT-AC (patient #2). The motion is even more complex with the lesions attached to the rigid structure in the thorax, e.g., the pleura near the ribcage (patient #10). No significant difference in terms of quantification results was observed for different thoracic regions; this may be due to the small number of lesions in each region.

Patients tend to spend more time towards the end-expiration period (Liu et al., 2009) and this can be reflected in the asymmetry of the respiratory signal. For each patient, this degree of asymmetry was represented by an integer, n, in Eqn. (1) and can be determined for each patient specifically according to their acquired respiratory signal by calculating the elapsed time ratio between expiration and inspiration. Thus, the sinusoidal function for interpolation is indeed semi-patient specific. One may suggest using the fully patient specific breathing signal for the interpolation function. However, preliminary data showed that the quantitative difference of the PET results of using these two functions were negligible. Also, the errors of determination of the PET and CT centroids were negligible, with ˜4% and ˜3% for CT and PET, respectively, for a 6 mm spherical lesion with 8:1 uptake ratio from a simulation study (data not shown). In this example, the PET_{IACT} was done after the PET_{HCT} as the positioning of the ABC device was not feasible for the standard whole-body PET protocol. Thus, concatenating the IACT into the standard whole-body HCT (Pan et al., 2006) was not a possible option with the setup in this example. The longer activity distribution time for thoracic PET acquisition may “artificially” enhance the lesion SUV (Basu et al., 2011) and overestimate the effectiveness of IACT. However, we found the average SUVₘₑₐₙ increase (32.9%) of the malignant lung tumors (n=9) was substantially higher than the increased value (20.5%) as suggested by Matthies et al. (2002) who evaluated dual time point FDG PET for pulmonary nodules, even the delay between 2nd and 1st PET scan was just 30 min as compared to ˜60 min in other dual time point studies. Also, all benign lung lesions (n=5) in this example showed obvious SUV increase in PET_{IACT} as compared to PET_{HCT}, although they usually do not demonstrate increased SUV for dual time point scans (Matthies et al., 2002). The lesion centroid differences between PET and associate CT also confirmed the effectiveness of IACT (Table II). The thoracic IACT can be incorporated into standard whole-body HCT directly to become a composite whole-body CT dataset for whole-body PET reconstruction (Pan et al., 2006) to rule out the potential delayed uptake effect. Besides, the IACT generation is automatically done with two registration processes, while gated 4D PET/CT may require multiple registrations. For CACT, there is no external respiratory monitoring device and breath-hold required for the acquisition, making it clinically more favorable. However, the dose reduction of IACT as compared to CACT is still substantial (0.38 mSv vs less than 1 mSv (Pan et al., 2013), 1.35 mSv (Pan et al., 2006), and 2.36 mSv (Gould et al., 2007)). The IACT was applied for PET/CT cardiac scans and showed substantial image quality improvement on the reconstructed images (Wu et al., 2010).

### Conclusion

An ABC prototype was built and the clinical feasibility of IACT-ABC method on patients with thoracic tumors was evaluated. It was shown that it potentially improved PET reconstructed image quality as compared to the conventional HCT, with reduced respiratory artifacts and spatial mismatch, increased SUV of the lesions and lowered radiation dose. IACT with the aid of ABC is a robust method for thoracic PET/CT AC and is promising to improve diagnostic accuracy.

### Example 2

### Clinical Setup

In this Example, two normal volunteers were recruited and images were acquired using a PET/CT scanner (Discovery VCT, GE Medical Systems, Milwaukee, Wis., USA). The patients were injected with 328 MBq and 406 MBq of ¹⁸F-FDG respectively and scanned 1 hour post injection. Thoracic PET data were acquired for 2 bed positions with 3 minutes per bed position. One standard helical CT (HCT), two extra end-inspiration and end-expiration breath-hold helical CTs were obtained using ABC for IACT were performed for each subject (FIG. 8). The standard HCT acquisition settings were: 120 kV, smart mA (range 30-150 mA) helical mode, 0.984:1 pitch, 0.5 s gantry rotation and total 4.4 s scan time. For IACT, two breath hold CTs were acquired at 120 kV, 10 mA helical modes, 0.984:1 pitch, 0.5 s gantry rotation time and a total of 4.4 s acquisition time for each scan.

### IACT Generation

B-spline, a deformable image registration algorithm, was applied to calculate the deformation vectors which includes lateral, anterior-posterior and inferior-superior displacement for each voxel on two CT volumes, i.e., end-inspiration and end-expiration phases obtained from ABC, based on the Insight Segmentation and Registration Toolkit (ITK) (WU et al., 2008). One CT image was chosen as the fixed image while the other was used as the moving image. A single rigid registration was conducted in the first step. Three stages of B-spline registration were performed later using multi-resolution method in the second step. The grid resolution of the control points improved and their grid-spacing decreased along different stages in this step. The deformation field was determined when the mean square error of the two CT images was smaller than some positive value ε in each resolution level. The forward deformation vector Φᵢₑ was calculated from end-inspiration phase #1 to end-expiration phase #7 and backward deformation vector Φₑᵢ was calculated from phase #7 to phase #1. For interpolation, an upper liver movement function in one respiratory cycle was used (Lujan et al., 2003):

$$z ( t ) = {z}_{o} - b {cos}^{2 n} ( \frac{π t}{τ} ) , \qquad Eqn . ( 1 )$$

where z(t)=position of organ at time t, zₒ=position at end-expiration, b=amplitude of motion, τ=period of motion, and n=degree of asymmetry (n=1 here).

To generate intermediate images, Φᵢₑ was divided based on z(t) to obtain interpolated deformation fields. Thus, we can generate interpolated phases #2, #3, #4, #5, #6 by warping original phase #1 based on Φᵢₑ (FIG. 4). Similarly, phases #8, #9, #10, #11 and #12 were warped from phase #7 based on Φₑᵢ. The final IACT was generated by averaging the interpolated and the 2 original phases.

### Data Analysis

The PET sinograms were reconstructed using OS-EM algorithm available on the GE VCT workstation. Attenuation corrections were conducted using HCT and IACT respectively. Their reconstructed PET images quality and associated radiation dose were compared and analyzed:
(i) Image profile: Besides visual assessment, an image profile was drawn vertically across the lung and the diaphragm to demonstrate the misalignment artifacts in the reconstructed images using different CT protocols.
(ii) Radiation dose: Estimated effective doses in mSv were calculated for different CT protocols and subjects.

### Results

From visual assessment, breathing artifacts were observed on the PET images with HCT-based AC but they were significantly reduced for those with IACT-based AC (FIG. 9). The quantitative profiles confirmed the potential difference between the 2 sets of PET reconstructed images (FIG. 10). The dose report suggested the IACT had an estimated dose of 0.38 mSv, reducing the dose up to 87% as compared with standard HCT (Table III).

TABLE III
Radiation dose for different CT protocols

| | HCT (mSv) | IACT (mSv) |
|---|---|---|
| Subject #1 | 2.83 | 0.38 |
| Subject #2 | 2.04 | 0.38 |

### Discussion and Conclusion

IACT used together with ABC provides improved PET image quality as compared to HCT with reduced radiation dose. IACT is feasible and robust in clinical practice with the aid of ABC and further improvement by consideration of specific patient's breathing condition is expected.

### Example 3

In this example, 4D Extended Cardiac Torso (XCAT) Phantom is used for realistically model the anatomy, activity distribution of a patient injected with ¹⁸F-FDG, and the respiratory motions. An analytical projector and OS-EM reconstruction algorithm provided by STIR (Software for Tomographic Image Reconstruction) was used for modelling a GE Discovery STE PET Scanner. The respiratory cycle was divided into 13 phases starting from the end-inspiration phase, and two maximum respiratory motion amplitudes of 2 cm and 3 cm were modeled. The attenuation maps representing CACT, IACT, HCTᵢₙ and HCTₑₓ are shown in FIG. 11 (a) to (d). Thoracic spherical lesions having diameters of 10 mm and 20 mm are placed at 4 different locations individually at lower left lung, lower right lung, middle right lung and upper right lung as shown in FIG. 12 (a) to (d). The target-to-background ratios (TBR) of 4:1 and 8:1 was used for respiratory amplitude of 2 cm while the TBR of 6:1 and 12:1 was used for respiratory amplitude of 3 cm. B-spline registration algorithm was used for calculating the deformation vectors for each voxel on HCTᵢₙ and HCTₑₓ and to generate the interpolated phases based on an empirical sinusoidal function. IACT was finally obtained by averaging the interpolated and HCTᵢₙ and HCTₑₓ. 2D TBR was calculated from known lesion region and chosen background region in PET reconstructed images using the formula:

$$TBR = \frac{{Mean}_{lesion}}{{Mean}_{background}}$$

The results for the simulation are shown in FIGS. 13 to 20.

It could be clearly demonstrated that:
1. PET_{IACT} more closely mimicked PET_{CACT} for all lesion specifications as compared to PET_{HCT}.
2. PET_{HCT-in} consistently showed under-estimation while PET_{HCT-ex} usually showed better TBR recovery.
3. The PET_{HCT} deviated from the PET_{IACT}/PET_{CACT} more for the lower level lesions, following by the middle level and upper level lesions.
4. TBRs of 10 mm lesions were more difficult to be recovered for all AC maps.
5. IACT works similarly for different lesion sizes uptake ratios and motion amplitudes.

### Example 4

In this example, 4D Extended Cardiac Torso (XCAT)

Phantom is used for realistically model the anatomy, activity distribution of a patient injected with ¹⁸F-FDG, and the respiratory motions. An analytical projector and OS-EM reconstruction algorithm provided by STIR (Software for Tomographic Image Reconstruction) was used for modelling a GE Discovery STE PET Scanner. The respiratory cycle was divided into 13 phases starting from the end-inspiration phase, and three maximum respiratory motion amplitudes of 2 cm, 3 cm and 4 cm were modeled. The attenuation maps used in this example are shown in FIG. 21. The cardiac defect placed at the inferior and lateral wall of the left ventricle was 2 cm longitudinally, circumferential 60°, 60% normal uptake as shown in FIG. 22. B-spline registration algorithm was used for calculating the deformation vectors for each voxel on HCTᵢₙ and HCTₑₓ and to generate the interpolated phases based on an empirical sinusoidal function. IACT was finally obtained by averaging the interpolated and HCTᵢₙ and HCTₑₓ. Bull's eye plots were generated from short axis images and circumferential profiles were measured at different apex-to-base distances. Regions-of-interest (ROIs) drawn on lesions and background area to calculate the intensity ratios, IR:

$$I R = \frac{{ROI}_{lesion}}{{ROI}_{background}}$$

The simulation results are shown in FIGS. 23 to 26 and Table IV.

It could be clearly demonstrated that:
1. The bull's eye plots of PET_{IACT} were more similar to PET_{CACT} from visual assessment.
2. The circumferential profiles showed that PET_{IACT} approached to PET_{CACT} while PET_{HCT-1} showed lower and PET_{HCT-8} showed higher intensity as compared to PET_{CACT}.
3. The IR differences of PET_{HCT-8} as compared to the phantom were up to ˜20%.

### Example 5

### Clinical Setup

In this Example, 4 male subjects aged 30-86 were recruited and images were acquired using a PET/CT scanner (Discovery VCT, GE Medical Systems, Milwaukee, Wis., USA). The patients were injected with 315 MBq to 428 MBq of ¹⁸F-FDG and scanned 1 hr post injection. Whole-body PET data were acquired for 7 bed positions and thoracic PET data were acquired for 2 bed positions at 3 minutes per bed position. The effective dose administered ranged from 7-10 mSv. One standard helical CT (HCT), two extra end-inspiration and end-expiration breath-hold helical CTs were obtained using ABC for IACT were performed for each subject. The standard free breathing whole body HCT acquisition settings were: 120 kV, smart mA (30-150 mA), 0.984 pitch, 8×2.5 mm collimation, 0.5 s CT gantry rotation, 15 s acquisition time. IACT was obtained from 2 separate thoracic HCTs with the aid of an active breathing controller (ABC). The two breath hold CTs were acquired at 120 kV, 10 mA helical modes, 0.984:1 pitch, 8×2.5 mm collimation, 0.5 s gantry rotation time and a total of 4.4 s acquisition time for each scan. Cine CT was acquired at 120 kV, 10 mA helical modes, 5.9 s cine duration.

The same IACT generation method used is the same as in Example 2. OS-EM image reconstruction was carried out with 2 iterations with 28 subsets for clinical data and attenuation correction was conducted with HCT, IACT and CACT as the attenuation map. The reconstructed images were visually assessed and quantified based on the bull's eye plots generated from short axis images. Circumferential profiles were measured at different apex-to-base distances on the bull's eye plots and doses from different CT protocols were also compared.

It could be clearly demonstrated that:
1. The bull's eye plots of PET_{IACT} were more similar to PET_{CACT} as compared to PET_{HCT} from visual assessment and circumferential profiles
2. Artifactual non-uniformity observed for PET_{HCT}
3. Estimated dose for IACT was reduced to 0.38 mSv for the thorax, by ˜84% compared to the use of HCT & CACT
4. With the aid of ABC, IACT is a robust and accurate low-dose CT protocol for cardiology applications

### Example 6

In this example, 4D Extended Cardiac Torso (XCAT) Phantom is used for realistically model the anatomy, activity distribution of a patient injected with ⁸²Rb, a cardiac perfusion tracer. An analytical projector was used for modelling a GE Discovery RX PET Scanner. The respiratory cycle was divided into 13 phases starting from the end-inspiration phase, and a maximum respiratory motion amplitudes of 2 cm was modeled. B-spline registration algorithm was used for calculating the deformation vectors for each voxel on HCTᵢₙ and HCTₑₓ and to generate the interpolated phases based on an empirical sinusoidal function. IACT was finally obtained by averaging the interpolated and HCTᵢₙ and HCTₑₓ. Bull's eye plots were generated from short axis images and circumferential profiles were measured at different apex-to-base distances.

The simulation results are shown in FIG. 30.

PET_{IACT} was shown to be more closely mimicked PET_{CACT} as compared to PET_{HCT}.

### REFERENCES

M. C. Adams et al., “A systematic review of the factors affecting accuracy of SUV measurements,” Am. J. Roentgenol. 195(2), 310-320 (2010).
M. Allen-Auerbach et al., “Standard PET/CT of the chest during shallow breathing is inadequate for comprehensive staging of lung cancer,” J. Nucl. Med. 47(2), 298-301 (2006).
A. M. Alessio et al., “Cine CT for attenuation correction in cardiac PET/CT,” J. Nucl. Med. 48(5), 794-801 (2007).
S. Basu et al., “Quantitative techniques in PET-CT imaging,” Curr. Med. Imaging Rev. 7(3), 216-233 (2011).
R. Boellaard et al., “FDG PET and PET/CT: EANM procedure guidelines for tumour PET imaging: Version 1.0,” Eur. J. Nucl. Med. Mol. Imaging 37(1), 181-200 (2010).
V. Boldea et al., “4D-CT lung motion estimation with deformable registration: Quantification of motion nonlinearity and hysteresis,” Med. Phys. 35(3), 1008-1018 (2008).
G. Bongartz et al., European Guidelines for Multislice Computed Tomography (European Commission, Brussels, Belgium, (2004).
L. I. Cervino et al., “The diaphragm as an anatomic surrogate for lung tumor motion,” Phys. Med. Biol. 54(11), 3529-3541 (2009).
E. Deniaud-Alexandre et al., “Impact of computed tomography and 18F-deoxyglucose coincidence detection emission tomography image fusion for optimization of conformal radiotherapy in non-small-cell lung cancer,” Int. J. Radiat. Oncol. Biol. Phys. 63(5), 1432-1441 (2005).
D. Didierlaurent et al., “The retrospective binning method improves the consistency of phase binning in respiratory gated PET/CT,” Phys. Med. Biol. 57(23), 7829-7841 (2012).
Y. E. Erdi et al., “The CT motion quantitation of lung lesions and its impact on PET-measured SUVs,” J. Nucl. Med. 45(8), 1287-1292 (2004).
H. J. Fayad et al., “Generation of 4-dimensional CT images based on 4-dimensional PET-derived motion fields,” J. Nucl. Med. 54(4), 631-638 (2013).
L. Fin et al., “Initial clinical results for breath-hold CT-based processing of respiratory gated PET acquisitions,” Eur. J. Nucl. Med. Mol. Imaging 35(11), 1971-1980 (2008).
G. W. Goerres et al., “Accuracy of image coregistration of pulmonary lesions in patients with non-small cell lung cancer using an integrated PET/CT system,” J. Nucl. Med. 43(11), 1469-1475 (2002).
K. L. Gould et al., “Frequent diagnostic errors in cardiac PET/CT due to misregistration of CT attenuation and emission PET images: A definitive analysis of causes, consequences, and corrections,” J. Nucl. Med. 48(7), 1112-1121 (2007).
N. Grotus et al., “Fully 4D list-mode reconstruction applied to respiratory-gated PET scans,” Phys. Med. Biol. 54(6), 1705-1721 (2009).
R. de Juan et al., “Clinical evaluation of a breathing protocol for PET/CT,” Eur. Radiol. 14(6), 1118-1123 (2004).
F. Lamare et al., “List-mode-based reconstruction for respiratory motion correction in PET using non-rigid body transformations,” Phys. Med. Biol. 52(17), 5187-5204 (2007).
C. Liu et al., “The impact of respiratory motion on tumor quantification and delineation in static PET/CT imaging,” Phys. Med. Biol. 54(24), 7345-7362 (2009).
C. Liu et al., “Quiescent period respiratory gating for PET/CT,” Med. Phys. 37(9), 5037-5043 (2010).
C. Loghin et al., “Common artifacts in PET myocardial perfusion images due to attenuation-emission misregistration: Clinical significance, causes, and solutions,” J. Nucl. Med. 45(6), 1029-1039 (2004).
A. E. Lujan at al., “A method for incorporating organ motion due to breathing into 3D dose calculations in the liver: Sensitivity to variations in motion,” Med. Phys., vol. 30, pp. 2643-2649, 2003.
K. Mah et al., “The impact of 18 FDG-PET on target and critical organs in CT-based treatment planning of patients with poorly defined non-small-cell lung carcinoma: A prospective study,” Int. J. Radiat. Oncol. Biol. Phys. 52(2), 339-350 (2002).
A. Martinez-Möller et al., “Artifacts from misaligned CT in cardiac perfusion PET/CT studies: Frequency, effects, and potential solutions,” J. Nucl. Med. 48(2), 188-193 (2007).
A. Matthies et al., “Dual time point 18F-FDG PET for the evaluation of pulmonary nodules,” J. Nucl. Med. 43(7), 871-875 (2002).
S. McQuaid et al., “Sources of attenuation-correction artefacts in cardiac PET/CT and SPECT/CT,” Eur. J. Nucl. Med. Mol. Imaging 35(6), 1117-1123 (2008).
S. J. McQuaid et al., “A novel method for incorporating respiratory-matched attenuation correction in the motion correction of cardiac PET-CT studies,” Phys. Med. Biol. 56(10), 2903-2915 (2011).
C. C. A. Nagel et al., “Phased attenuation correction in respiration correlated computed tomography/positron emitted tomography,” Med. Phys. 33(6), 1840-1847 (2006).
S. A. Nehmeh et al., “Four-dimensional (4D) PET/CT imaging of the thorax,” Med. Phys. 31(12), 3179-3186 (2004).
S. A. Nehmeh et al., “Deep-inspiration breath-hold PET/CT of the thorax,” J. Nucl. Med. 48(1), 22-26 (2007).
S. A. Nehmeh et al., “Respiratory motion in positron emission tomography/computed tomography: A review,” Semin. Nucl. Med. 38(3), 167-176 (2008).
J. A. Nye et al., “Minimizing artifacts resulting from respiratory and cardiac motion by optimization of the transmission scan in cardiac PET/CT,” Med. Phys. 34(6), 1901-1906 (2007).
M. M. Osman et al., “Respiratory motion artifacts on PET emission images obtained using CT attenuation correction on PET-CT,” Eur. J. Nucl. Med. Mol. Imaging 30(4), 603-606 (2003).
T. Pan et al., “Attenuation Correction of PET Images with Respiration-Averaged CT Images in PET/CT,” J. Nucl. Med., vol. 46, pp. 1481-1487, Sep. 1 2005.
T. Pan et al., “Attenuation correction of PET cardiac data with low-dose average CT in PET/CT,” Med. Phys. 33(10), 3931-3938 (2006).
T. Pan et al., “Attenuation correction strategies for positron emission tomography/computed tomography and 4-dimensional positron emission tomography/computed tomography,” PET Clin. 8(1), 37-50 (2013).
M. Rajaram et al., “Cardiac PET/CT misregistration causes significant changes in estimated myocardial blood flow,” J. Nucl. Med. 54(1), 50-54 (2013).
D. Sarrut et al., “Simulation of four dimensional CT images from deformable registration between inhale and exhale breath-hold CT scans,” Med. Phys. 33(3), 605-617 (2006).
G. K. von Schulthess et al., “Integrated PET/CT: Current applications and future directions,” Radiology 238(2), 405-422(2006).
S. Senan et al., “Literature-based recommendations for treatment planning and execution in high-dose radiotherapy for lung cancer,” Radiother. Oncol. 71(2), 139-146 (2004).
Y. Seppenwoolde et al., “Precise and real-time measurement of 3D tumor motion in lung due to breathing and heartbeat, measured during radiotherapy,” Int. J. Radiat. Oncol. Biol. Phys. 53(4), 822-834 (2002).
T. Sun et al., “Techniques for respiration-induced artifacts reductions in thoracic PET/CT,” Quant. Imaging Med. Surg. 2, 46-52 (2012).
R. G. Wells et al., “Single-phase CT aligned to gated PET for respiratory motion correction in cardiac PET/CT,” J. Nucl. Med. 51(8), 1182-1190 (2010).
Z. Wu et al., “Evaluation of deformable registration of patient lung 4DCT with subanatomical region segmentations,” Med. Phys., vol. 35, pp. 775-781, 2008.
T.-H. Wu et al., “Low-dose interpolated average CT for attenuation correction in cardiac PET/CT,” Nucl. Instrum. Meth. A 619(1-3), 361-364 (2010).
T. Xia et al., “Ultra-low dose CT attenuation correction for PET/CT,” Phys. Med. Biol. 57(2), 309-328 (2012).
T. S. Yoo et al., “Engineering and algorithm design for an image processing Api: A technical report on ITK—The Insight Toolkit,” Stud. Health Technol. Inform. 85, 586-592 (2002).

What is claimed is:

1. A method of attenuation correction for emission computed tomography (ECT) reconstruction, comprising:
i. generating Interpolated Average CT (IACT) image of a subject based on one or more CT images acquired during momentary suspension of breathing motion at specific phases in the breathing cycle of a subject by means of a breathing control device;
ii. acquiring an ECT image from the same section of the subject; and
iii. correcting attenuation in said ECT image of step (ii) by using said IACT image generated previously from step (i) as an attenuation map in the ECT reconstruction that comprises the use of a reconstruction algorithm, thereby resulting in reconstructed ECT images with attenuation correction.

2. The method of claim 1, wherein said emission computed tomography is Positron Emission Tomography (PET) or Single Photon Emission Computed Tomography (SPECT).

3. The method of claim 1, wherein said specific phase in the breathing cycle comprises one or more of end-expiration phase, end-inspiration phase and any one of the mid-respiratory phases.

4. The method of claim 1, wherein said image reconstruction algorithm is a 2-D, 2.5-D, 3-D or 4-D image reconstruction algorithm selected from the group consisting of Ordered Subsets Expectation Maximization (OS-EM) image reconstruction algorithm, Filtered Back Projection (FBP) method, Maximum Likelihood Expectation Maximization (ML-EM), Maximum a Posteriori Expectation Maximization (MAP-EM) with different priors, Maximum a Posteriori Expectation Maximization based on a One-Step-Late algorithm (MAP-EM (OSL)) with different priors, Row Action maximum Likelihood Algorithm (RAMLA), 3D ReProjection (3DRP), Single Slice Rebinning (SSRB), Fourier Rebinning (FORE), Fourier Rebinning with 2D algorithm (FORE+2D), and Fourier Rebinning with Average-Weighted OS-EM Expectation Maximization (FORE+AWOS-EM).

5. The method of claim 1, wherein said subject has one or more lesions or no lesions in the thoracic cavity.

6. The method of claim 1, wherein said reconstructed image is used in assessing cardiac viability, myocardial perfusion, or presence or quantification of lesions in said subject.

7. The method of claim 1, wherein said Interpolated Average CT image is generated by a method comprising:
a. suspending breathing motion of said subject momentarily at said one or more specific phases in a breathing cycle by means of said breathing control device;
b. acquiring one or more CT images of said subject when the breathing motion is suspended by said breathing control device;
c. obtaining a deformation matrix of said CT images of step (b) by deformable image registration;
d. interpolating between the images of step (b) to obtain intermediate images base on said deformation matrix of step (c); and
e. generating Interpolated Average CT images by averaging the intensity of the images of step (b) and the intermediate images of step (d).

8. The method of claim 7, wherein said deformable image registration of step (c) is carried out by B-spline algorithm or optical flow algorithm.

9. The method of claim 7, wherein said interpolation of step (d) is linear or nonlinear interpolation.

10. The method of claim 9, wherein said nonlinear interpolation is based on the movement function of an internal organ during respiration, defined by:
$$z ( t ) = {z}_{o} - b {cos}^{2 n} ( \frac{π t}{τ} ) ,$$
where
z(t)=position of said organ at time, t;
zₒ=organ position at end-expiration;
b=amplitude of motion;
τ=period of motion;
n=degree of symmetry which depends on the patient-specific respiratory signal.

11. The method of claim 10, wherein said internal organ is liver or diaphragm of said subject.

12. The method of claim 9, wherein said nonlinear interpolation is based on respiratory signals generated by a computer simulation model.

13. The method of claim 12, wherein said computer simulation model is a 4-Dimensional Non-uniform Rational B-spline (NURBS) based Cardiac-Torso (XCAT) phantom.

14. The method of claim 9, wherein said nonlinear interpolation is based on read-in patient-specific respiratory signal.

15. The method of claim 7, wherein said breathing control device of step (a) is an active breathing controller.

16. The method of claim 7, wherein said CT image of step (b) is acquired using helical CT or cine CT.

17. The method of claim 7, wherein said CT image of step (b) is acquired at a reduced radiation dose.

18. The method of claim 17, wherein said radiation dosage is reduced up to 85% as compared to conventional helical CT.

19. A system for generating Interpolated Average CT image of a subject, comprising:
i. an active breathing controller (ABC) for momentarily suspending breathing motion of a subject at one or more specific phases in a breathing cycle, said ABC comprises a flow sensor, a valve, a microcontroller and an airtube system, wherein said subject breathes through the airtube system;
ii. a computing device comprising a program for identifying one or more specific phases in the breathing cycle based on breathing flow rate data, said device is configured to control the valve in the ABC;
iii. a CT scanner, wherein said CT scanner acquires one or more CT images when said subject's breathing motion is momentarily suspended by said ABC.

20. The system of claim 19, wherein said airtube system comprises a mask or mouth piece for said subject to breathe through.

21. The system of claim 19, wherein said flow sensor measures breathing flow rate of said subject breathing through the airtube system.

22. The system of claim 19, wherein said microcontroller receives breathing flow rate signal from the flow sensor and sends the signal to the computing device.

23. The system of claim 19, wherein said computing device closes the valve in the ABC to suspend breathing motion of said subject when one or more specific phases in the breathing cycle is identified manually or automatically by said program.

24. The system of claim 19, wherein CT images for generating Interpolated Average CT image are acquired manually or automatically when the breathing motion of said subject is suspended.

25. The system of claim 19, wherein said CT scanner is coupled with a PET or a SPECT scanner.
